SEQUENCE LISTING (29 sequences)

SEQ ID NO: 1 - protein, 18 aa, Artificial Sequence
  Synthetic peptide
MASRAAGLAARLARLALR

SEQ ID NO: 2 - protein, 19 aa, Artificial Sequence
  Synthetic peptide
MASRAAGLAARLARLALRA

SEQ ID NO: 3 - protein, 20 aa, Artificial Sequence
  Synthetic peptide
MASRAAGLAARLARLALRAL

SEQ ID NO: 4 - protein, 17 aa, Artificial Sequence
  Synthetic peptide
ASRAAGLAARLARLALR

SEQ ID NO: 5 - protein, 18 aa, Artificial Sequence
  Synthetic peptide
MVSRAAGLAARLARLALR

SEQ ID NO: 6 - protein, 19 aa, Artificial Sequence
  Synthetic peptide
MVSRAAGLAARLARLALRA

SEQ ID NO: 7 - protein, 20 aa, Artificial Sequence
  Synthetic peptide
MVSRAAGLAARLARLALRAL

SEQ ID NO: 8 - protein, 18 aa, Artificial Sequence
  Synthetic peptide
MASRAAGLARRLARLARR

SEQ ID NO: 9 - protein, 19 aa, Artificial Sequence
  Synthetic peptide
MASRAAGLARRLARLARRA

SEQ ID NO: 10 - protein, 20 aa, Artificial Sequence
  Synthetic peptide
MASRAAGLARRLARLARRAL

SEQ ID NO: 11 - protein, 18 aa, Artificial Sequence
  Synthetic peptide
MAARAAGLAARLAALALR

SEQ ID NO: 12 - protein, 19 aa, Artificial Sequence
  Synthetic peptide
MAARAAGLAARLAALALRA

SEQ ID NO: 13 - protein, 20 aa, Artificial Sequence
  Synthetic peptide
MAARAAGLAARLAALALRAL

SEQ ID NO: 14 - protein, 15 aa, Artificial Sequence
  Synthetic peptide
SRAAGLAARLARLAL

SEQ ID NO: 15 - protein, 29 aa, Artificial Sequence
  Synthetic peptide
MGECVRGRCPSGMCCSQFGYCGKGPKYCG

SEQ ID NO: 16 - protein, 18 aa, Artificial Sequence
  Synthetic peptide
MASRAARLAARLARLALR

SEQ ID NO: 17 - protein, 19 aa, Artificial Sequence
  Synthetic peptide
MASRAARLAARLARLALRA

SEQ ID NO: 18 - protein, 17 aa, Artificial Sequence
  Synthetic peptide
ASRAARLAARLARLALR

SEQ ID NO: 19 - protein, 18 aa, Artificial Sequence
  Synthetic peptide
MVSRAARLAARLARLALR

SEQ ID NO: 20 - protein, 19 aa, Artificial Sequence
  Synthetic peptide
MVSRAARLAARLARLALRA

SEQ ID NO: 21 - protein, 20 aa, Artificial Sequence
  Synthetic peptide
MVSRAARLAARLARLALRAL

SEQ ID NO: 22 - protein, 18 aa, Artificial Sequence
  Synthetic peptide
MASRAARLARRLARLARR

SEQ ID NO: 23 - protein, 19 aa, Artificial Sequence
  Synthetic peptide
MASRAARLARRLARLARRA

SEQ ID NO: 24 - protein, 20 aa, Artificial Sequence
  Synthetic peptide
MASRAARLARRLARLARRAL

SEQ ID NO: 25 - protein, 18 aa, Artificial Sequence
  Synthetic peptide
MAARAARLAARLAALALR

SEQ ID NO: 26 - protein, 19 aa, Artificial Sequence
  Synthetic peptide
MAARAARLAARLAALALRA

SEQ ID NO: 27 - protein, 20 aa, Artificial Sequence
  Synthetic peptide
MAARAARLAARLAALALRAL

SEQ ID NO: 28 - protein, 15 aa, Artificial Sequence
  Synthetic peptide
SRAARLAARLARLAL

SEQ ID NO: 29 - protein, 13 aa, Artificial Sequence
  Synthetic peptide
RLARLARRLARLA

(19) United States
(12) Patent Application Publication
(10) Pub. No.: US 2012/0003119 A1
(43) Pub. Date: Jan. 5, 2012

(54) ANTIMICROBIAL PEPTIDES

(75) Inventors: William A. Powell, Syracuse, NY (US); Charles A. Maynard, Syracuse, NY (US)

(73) Assignee: The Research Foundation of State University of New York, Albany, NY (US)

(21) Appl. No.: 12/673,815

(22) PCT Filed: Aug. 15, 2008
(86) PCT No.: PCT/US08/73226

§ 371 Date: Jan. 20, 2011

Related U.S. Application Data
(60) Provisional application No. 60/965,253, filed on Aug. 17, 2007.

(51) Int. Cl. A61L 2/16; C07K 7/08; C07K 14/00; A61K 38/10; A61K 38/16

(52) U.S. Cl. 422/28; 514/2.3; 514/2.4; 514/2.8; 530/326; 530/324; 530/327

(57) ABSTRACT

The present invention is directed to a method for disinfecting or sterilizing food, particularly, fresh produce, fruits and vegetables, by applying antimicrobial polypeptides (AMP). The AMP used in the present invention consists of from 13 to 20 amino acids and has an amphipathic alpha helix structure, wherein 3 or more of the amino acids form a positively charged domain extending axially along the alpha helix.

### GOVERNMENT INTERESTS

This invention was made with government support under the United States Department of Agriculture Grant Nos. USDA #96 CRMS 06102 USDA #CSRSOD-1088 and USDA #95373032083. The Government has certain rights in the invention.

### FIELD OF THE INVENTION

The present invention relates to methods for minimizing and the prevention of microbial contaminations. Particularly, the present invention provides methods of reducing, eliminating or preventing microbial contaminations in fresh produce and vegetables by using antimicrobial polypeptides as a disinfectant.

### BACKGROUND OF THE INVENTION

Throughout this application various publications are referenced, many in parenthesis. Full citations for these publications are provided at the end of the Detailed Description of the Invention. The disclosures of these publications in their entireties, as well as those of U.S. patents referenced herein, are hereby incorporated by reference in the present application.

Fresh produce and vegetables are important to the health and well being of the consumer in the U.S. and all over the world. Consumers in the U.S. once enjoy one of the safest supplies of fresh produce in the world. However, over the last several years, the detection of outbreaks of foodborne illness associated with both domestic and imported fresh fruits and vegetables has increased. For example, in September 2006, the United States experienced a large multi-state outbreak of E. coli infections linked to fresh spinach consumption (Centers for Disease Control and Prevention, MMWR Dispatch: Ongoing Multistate Outbreak of Escherichia coli serotype O157:H7 Infections Associated with Consumption of Fresh Spinach—United States, September 2006, http://www.cdc.gov/mmwr/preview/mmwrhtml/mm55d926a1.htm (last visited Mar. 12, 2007)). In the outbreak that spanned 26 states, within just one month there were a total of 199 cases of illness reported to the Centers for Disease Control and Prevention (CDC), resulting in 102 people being hospitalized and three deaths. This recent outbreak of E. coli infections related to spinach has brought national attention to the fact that fresh green vegetables can, as equally as ground beef, be contaminated with E. coli and other disease-causing bacteria, and illustrated the need for better control measures in the industry to prevent or reduce microbial contamination of fresh produce. While ground beef has been the traditional vehicle for food borne outbreaks caused by E. coli, produce-associated outbreaks are not unknown and in fact, since 1991, a significant number of E. coli outbreaks have been traced to fresh produce such as lettuce, sprouts, cabbage, apple cider and apple juice. Out of the 183 food borne outbreaks of E. coli that occurred during 1982-2002, there were 38 outbreaks associated with fresh produce.

The main risk for leafy green vegetables is due to the fact that they are ready-to-eat products consumed in the raw state, meaning that the contaminating bacteria found in the food will not be reduced by cooking and thus little can be done to prevent illness. While the E. coli contamination of food can occur at any stage from farm to table, at least half of the reported outbreaks were due to produce already contaminated before purchase. These produce items can become contaminated at the farm from manure or through irrigation water that has been contaminated. The contamination may also occur during processing due to contaminated equipment or poor handling practices by employees or farmers. Contamination can also occur through transport and storage equipment. Produce items can be contaminated anywhere in the supply chain. See FIGS. 1 and 2.

Spinach can be exposed to microorganisms from many different sources, and so far no one disinfectant technology can protect from every source. Worker education and safety procedures can protect against some exposure routes, but other routes can be very difficult to monitor and protect against.

Antimicrobial agents are compounds that reduce the microbial population on surfaces, foods, and other objects. The use of these agents can be important in the food industry because they serve as disinfectants or sanitizers. Other disinfectants or sanitizers include: chlorine dioxide, ozonation, quaternary ammonium compounds, phenolics, peracetic acid, sodium hypochlorite, amphoteric compounds, and ultraviolet light radiation.

A steadily increasing interest is being focused on defense peptides produced by a variety of organisms (Cornelissen and Melchers 1993). These peptides or their analogs have the potential as a new source for disease resistant genes. Most of these small, lytic, antimicrobial peptides have been placed into four chemically distinct groups: the magainins, the cecropins, the defensins, and the proline-rich peptides (Agerberth et al. 1991). Antimicrobial peptide (AMP) (e.g., ESF12 peptide) technology is also described in the U.S. Pat. No. 5,856,127, which is hereby incorporated by reference in its entirety. The technology for disease prevention has been used in certain species of trees.

The cecropins, first isolated from the cecropia moth, but recently from many insects, range from 26-37 amino acids in length. Their structure includes two α-helical regions, one amphiphilic and one hydrophobic, joined by a hinge region (Christensen et al. 1988). The cecropins are thought to produce single-channel conductances in lipid bilayers such as in a cell membrane (Wade et al. 1990). Most of these peptides described to date also demonstrate a specificity to microorganisms. One exception is melittin, isolated from bee venom. This peptide is very lytic to both microorganisms and animal red blood cells. The specificity of melittin can be altered by inverting the α-helical regions or by producing cecropin A/melittin hybrids (Boman et al. 1989). Amino acid omission studies on melittin showed that deletions in the α-helical regions decreased hemolytic activity but deletions in the “hinge” region did not (Blondelle and Houghten 1991).

The magainins, from the skin of the African clawed frog (Xenopus laevus), are some of the smallest natural antimicrobial peptides yet discovered, ranging from 21-27 amino acids in length (Zasloff 1987 and Bevins and Zasloff 1990). These peptides form an amphipathic, single α-helix which can span a cell membrane. It is hypothesized that these molecules form ion channels in the microbial cell's membrane which the cell cannot control, eventually leading to lysis of the cell. The α-helix is essential for activity and changes in the amino acid sequence which stabilize this helical structure enhance the molecule's lytic activity against selected bacteria (Chen et al. 1988).

The magainins are of interest because of their ability to lyse bacterial and yeast cells but not animal cells (Soravia et al. 1988), suggesting good potential for use in agricultural and forest plant species. Different peptides from this group also demonstrated synergistic effects. When the peptide PGLa was combined with either magainin I or magainin II in a 1:1 molar ratio, the antimicrobial activity increased 20-50 fold. Interestingly, alone these peptides had no hemolytic effect but in combination they exhibited the ability to lyse a variety of eukaryotic cells (Bevins and Zasloff 1990). This complementation demonstrated synergistic effects which should be considered when studying combinations of these types of peptides.

Although all the peptides in the magainins group have similar amphipathic α-helical structures, small differences in their amino acid sequences result in different antimicrobial activity. One example of this difference can be discerned by comparing the reported activities of PGLa and XPF (Soravia et al. 1988). These two peptides are very similar in terms of structure. When tested against the bacteria Pseudomonas aeruginosa, PGLa was 4-5 times more active than XPF. When these two were tested against the yeast Candida albicans; however, XPF was 2-2.5 times more effective than PGLa. Similar differences could also be seen among different species of bacteria (Soravia et al. 1988). Even a three amino acid substitution in (Ala.sup.8,13,15)magainin II, which is reported to increase helical stability, caused a change in activities on bacterial strains (Chen et al. 1988). It has been observed that these structural changes increased magainin activity against bacteria but had the opposite effect against selected filamentous fungi. This indicated that the ratios of activity between different organisms could potentially be manipulated.

U.S. Pat. No. 5,856,127 provides an isolated nucleic acid molecule encoding a polypeptide. The polypeptide is antimicrobial, consists of from 15 to 20 amino acids, and has an amphipathic alpha helix structure. Three or more of the amino acids of the polypeptide form a positively charged domain extending axially along the alpha helix.

There is a need of effective disinfectants, sanitizers, or sterilization agents for use to inhibit the growth of, or kill, harmful microbes. Such microbes can be found on the surface of fresh produce, fruits and vegetables; in potable water; or in wounds, for example.

### SUMMARY OF INVENTION

The present invention recognizes that antimicrobial peptides, such as those provided by U.S. Pat. No. 5,856,127 and U.S. Pat. No. 5,643,876, can be used as a disinfectant or sterilization agent in the agricultural product and food-handling industries. The present invention recognizes that antimicrobial peptides provide high effectiveness against a broad spectrum of harmful microorganisms while exhibiting none of the harmful characteristics associated with several of the alternative technologies. These antimicrobial peptides do not need to be rinsed off after application. Moreover, antimicrobial peptides used in the method of the present invention have the ability to protect produce from harmful microorganisms from agricultural production and handling to consumption without altering the taste or quality of the produce.

In one aspect, the present invention is direct to a method for disinfecting or sterilizing an object comprising applying to the object at least one antimicrobial polypeptide (AMP), preferably, the amino acid sequence of the AMP is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28; and SEQ ID NO:29.

The present invention contemplates combinations of two or more AMPs will be effective in the described methods.

In another aspect, the present invention is directed to a method for disinfecting or sterilizing an object comprising applying to the object at least one AMP, wherein the amino acid sequence of the AMP is a polypeptide comprising or including an amino acid sequence of 13+q amino acid residues. In this polypeptide, residues number n, n+7, n+10, and n+14 are positively charged amino acids. In addition, at least one of residues number n, n+7, n+10, and n+14 is arginine, and the remaining amino acid residues are nonpolar amino acids or uncharged polar amino acids. n is an integer from 1 to 1+q, and q is 0, 1, 2, 3, 4, 5, 6, or 7.

In still another aspect, the present invention is directed to a method for disinfecting or sterilizing an object comprising applying to the object at least one AMP, wherein the amino acid sequence of the AMP is a polypeptide comprising or including an amino acid sequence of 13+q amino acid residues. The residues number n, n+6, n+7, n+10, n+13, and n+14 are positively charged amino acids, and the remaining amino acid residues are nonpolar amino acids or uncharged polar amino acids. n is an integer from 1 to 1+q, and q is 0, 1, 2, 3, 4, 5, 6, or 7.

In yet another aspect, the present invention is directed to a method for disinfecting or sterilizing an object comprising applying to the object at least one AMP, wherein the amino acid sequence of the AMP is a polypeptide comprising or including an amino acid sequence of 13+q amino acid residues. Residues number n, n+7, and n+14 are positively charged amino acids, and the remaining amino acid residues are nonpolar amino acids. n is an integer from 1 to 1+q, and q is 0, 1, 2, 3, 4, 5, 6, or 7.

In still yet another aspect, the present invention is directed to a method for disinfecting or sterilizing an object comprising applying to the object at least one AMP, wherein the amino acid sequence of the AMP is a polypeptide comprising or including an amino acid sequence of 13+q amino acid residues. The residues number n, n+7, and n+10 are positively charged amino acids, and the remaining amino acid residues are nonpolar amino acids. n is an integer from 1 to 5+q, and q is 0, 1, 2, 3, 4, 5, 6, or 7.

In another aspect, the present invention is directed to a method for disinfecting or sterilizing an object comprising applying to the object at least one AMP, wherein the amino acid sequence of the AMP is a polypeptide comprising an amino acid sequence of 13+q amino acid residues, wherein residues number n, n+3, n+7, n+10, and n+14 are positively charged amino acids, wherein at least one of residues number n, n+3, n+7, n+10, and n+14 is arginine, wherein remaining amino acid residues are nonpolar amino acids or uncharged polar amino acids, wherein n is an integer from 1 to 1+q, and wherein q is 0, 1, 2, 3, 4, 5, 6, or 7;

In still another aspect, the present invention is directed to a method for disinfecting or sterilizing an object comprising applying to the object at least one AMP, wherein the amino acid sequence of the AMP is a polypeptide comprising an amino acid sequence of 13+q amino acid residues, wherein residues number n, n+3, n+6, n+7, n+10, n+13, and n+14 are positively charged amino acids, wherein remaining amino acid residues are nonpolar amino acids or uncharged polar amino acids, and wherein n is an integer from 1 to 1+q, and wherein q is 0, 1, 2, 3, 4, 5, 6, or 7;

In still yet another aspect, the present invention is directed to a method for disinfecting or sterilizing an object comprising applying to the object at least one AMP, wherein the amino acid sequence of the AMP is a polypeptide comprising an amino acid sequence of 13+q amino acid residues, wherein residues number n, n+3, n+7, and n+14 are positively charged amino acids, wherein remaining amino acid residues are nonpolar amino acids, wherein n is an integer from 1 to 1+q, and wherein q is 0, 1, 2, 3, 4, 5, 6, or 7.

In a still further aspect, the present invention is directed to a method for disinfecting or sterilizing an object comprising applying to the object at least one AMP, wherein the amino acid sequence of the AMP is a polypeptide comprising an amino acid sequence of 13+q amino acid residues, wherein residues number n, n+3, n+7, and n+10 are positively charged amino acids, wherein remaining amino acid residues are nonpolar amino acids, wherein n is an integer from 1 to 5+q, and wherein q is 0, 1, 2, 3, 4, 5, 6, or 7.

In a particular aspect of the present invention, the polypeptide has a methionine residue as an N-terminal amino acid. In another particular aspect, the positively charged amino acids are the same or different and include, but are not limited to, lysine, arginine, and histidine. The nonpolar amino acids are the same or different and include, but are not limited to, alanine, valine, leucine, isoleucine, glycine, cysteine, phenylalanine, tryptophan, and methionine.

In one aspect, the AMP used in the present invention includes a polypeptide consisting of from 10 to 20 amino acids and preferably from 13 to 18 amino acids. The polypeptide is an antimicrobial and has an amphipathic alpha helix structure. Three or more of the amino acids of the polypeptide form a positively charged domain extending axially along the alpha helix.

In another aspect, the AMP used in the present invention includes a polypeptide comprising an amino acid sequence of 13+q amino acid residues. The residues number n, n+7, n+10, and n+14 are positively charged amino acids, and at least one of amino acids number n, n+7, n+10, and n+14 is arginine, and the remaining amino acids are nonpolar amino acids or uncharged polar amino acids. n is an integer from 1 to 1+q, and q is 0, 1, 2, 3, 4, 5, 6, or 7.

In still another aspect, the AMP used in the present invention includes a polypeptide comprising an amino acid sequence of 13+q amino acid residues. The residues number n, n+6, n+7, n+10, n+13, and n+14 are positively charged amino acids, and the remaining amino acid residues are nonpolar amino acids or uncharged polar amino acids. n is an integer from 1 to 1+q, and q is 0, 1, 2, 3, 4, 5, 6, or 7.

In yet another aspect, the AMP used in the present invention includes a polypeptide comprising an amino acid sequence of 13+q amino acid residues is provided. The residues number n, n+7, and n+14 are positively charged amino acids, and the remaining amino acid residues are nonpolar amino acids. n is an integer from 1 to 1+q, and q is 0, 1, 2, 3, 4, 5, 6, or 7.

In still yet another aspect, the AMP used in the present invention includes a polypeptide comprising an amino acid sequence of 13+q amino acid residues. The residues number n, n+7, and n+10 are positively charged amino acids, and the remaining amino acid residues are nonpolar amino acids. n is an integer from 1 to 5+q, and q is 0, 1, 2, 3, 4, 5, 6, or 7.

In a further aspect, the AMP used in the present invention includes a polypeptide comprising an amino acid sequence of 13+q amino acid residues, wherein residues number n, n+3, n+7, n+10, and n+14 are positively charged amino acids, wherein at least one of residues number n, n+3, n+7, n+10, and n+14 is arginine, wherein remaining amino acid residues are nonpolar amino acids or uncharged polar amino acids, wherein n is an integer from 1 to 1+q, and wherein q is 0, 1, 2, 3, 4, 5, 6, or 7.

In a still further aspect, the AMP used in the present invention includes a polypeptide comprising an amino acid sequence of 13+q amino acid residues, wherein residues number n, n+3, n+6, n+7, n+10, n+13, and n+14 are positively charged amino acids, wherein remaining amino acid residues are nonpolar amino acids or uncharged polar amino acids, and wherein n is an integer from 1 to 1+q, and wherein q is 0, 1, 2, 3, 4, 5, 6, or 7.

In yet another aspect, the AMP used in the present invention includes a polypeptide comprising an amino acid sequence of 13+q amino acid residues, wherein residues number n, n+3, n+7, and n+14 are positively charged amino acids, wherein remaining amino acid residues are nonpolar amino acids, wherein n is an integer from 1 to 1+q, and wherein q is 0, 1, 2, 3, 4, 5, 6, or 7.

In a further aspect, the AMP used in the present invention includes a polypeptide comprising an amino acid sequence of 13+q amino acid residues, wherein residues number n, n+3, n+7, and n+10 are positively charged amino acids, wherein remaining amino acid residues are nonpolar amino acids, wherein n is an integer from 1 to 5+q, and wherein q is 0, 1, 2, 3, 4, 5, 6, or 7.

The object to be disinfected or sterilized includes, but is not limited to, fresh produce, fruits or vegetables, or a surface area for fresh produce, fruits or vegetables.

Particularly, the vegetable is green leafy vegetable, such as spinach.

The microbes encompassed by the present invention include, but are not limited to, bacteria and fungus, particularly, E. coli and Salmonella.

In yet another aspect of the present invention, the AMP is applied to the object by any means suitable for disinfection and/or sterilization, including, but not limited to, spraying, washing, rinsing, wiping, dusting, or dipping.

A kit for disinfecting or sterilizing fresh produce, fruits or vegetables comprising at least one AMP and a tool for suitable application is also contemplated by the present invention. The tool can be any suitable means for application of AMP, such as a sprayer, duster, or wiping tissue.

### BRIEF DESCRIPTION OF THE DRAWING

These and other features and advantages of this invention will be evident from the following description of preferred embodiments when read in conjunction with the accompanying drawing in which:

FIG. 1 shows where microbial contamination can specifically occur and identifies the current practices in the food processing industry to control such contamination for the farm-to-table supply chain for lettuce and leafy greens.

FIG. 2 illustrates how leafy green vegetables pass through several stages including production and harvesting, post harvest handling, fresh-cut/value added operations, distribution and end user handling. Contamination can occur at each of these steps in the process, and once contaminated, it is difficult to remove or kill microorganisms.

### DETAILED DESCRIPTION OF THE INVENTION

The present invention is directed to a method for disinfecting or sterilizing an object by applying to the object with at least one antimicrobial polypeptide (AMP). The invention also contemplates isolated antimicrobial polypeptides.

A “disinfectant” or “sterilization agent” as used in the present application is defined as a product which is capable of completely destroying at least 50%, preferably, 90%, more preferably 99% of the microbes, or even more preferably, all the microbes within a short period of time, preferably, in 10 minutes, more preferably, in 1 minute, even more preferably, in 30 seconds. The disinfectants or sterilization agents of the present invention can be used either as a water wash to be applied directly onto food or as a surface cleaner. By “object” is meant e.g. the surface of a fruit or vegetable, a wound surface, a wound dressing, water, or any surface of anything susceptible to microbe growth or colonization or having microbes extant thereon. For example, the invention contemplates application of the disclosed AMP alone or in combination of two or more, to countertops, eating surfaces, bathroom surfaces, such as bathtubs, showers, sinks, toilets and the like.

Polypeptide chains form by condensation of two or more amino acid residues. In this process, the acid end of a first amino acid reacts with the amino end of a second amino acid, eliminating water, and forming a peptide bond in which the acid carbon becomes covalently bonded to the amine nitrogen. The resulting dipeptide, having both an acid as well as an amine terminus, can go on to react with a third amino acid, at either its carboxyl or amino end, to form a tripeptide. Further repetition of the process produces a polypeptide. Like the dipeptide and tripeptide, the polypeptide has an acid group at one end and an amine group at the other end. These ends are commonly referred to as the C-terminus (carboxy terminus) and the N-terminus (amino terminus) of the polypeptide.

Individual extended polypeptide chains are conformationally unstable and frequently either assume contracted helical configurations or aggregate side by side to form sheet-like structures. The driving force that leads to either of these two alternatives is the strong tendency of the carbonyl and imino groups that flank the peptide bonds to form non-covalent, hydrogen bonds. When the carbonyl group next to one peptide bond hydrogen-bonds to an imino group flanking another peptide bond several residues removed from it on the same chain, a highly repetitive, regular polypeptide conformation results. This folding pattern causes the polypeptide to assume a secondary structure known as the alpha helix. The likelihood that a particular polypeptide will form an alpha helix can be predicted from the polypeptide's primary amino acid sequence using, for example, Garnier-Robson (Garnier et al. 1978) or Chou-Fasman (Chou and Fasman 1978) algorithims within a Lasergene or PROTEAN program (DNASTAR, Inc., Madison, Wis.).

As used herein to describe the alpha helix of the polypeptide of the present invention, amphipathic means that the amino acid residues of the polypeptide are aligned three dimensionally to form both hydrophilic and hydrophobic regions. Preferably the polypeptide contains an amphipathic alpha helix structure as predicted by the Eisenberg Moment (Eisenberg et al. 1984).

As used herein, two or more positively charged amino acid residues of the polypeptide, when located in spatial proximity to one another, can form a positively charged domain. The spatial proximity can be achieved when the two positively charged amino acids are consecutive (in terms of primary structure). In this case, the positively charged domain extends circumferentially around the alpha helix. Alternatively, the spatial proximity of two or more positively charged amino acids can be achieved when the two residues lie in adjacent (or nearly adjacent) coils of the alpha helix. In this case, the positively charged domain extends axially, that is, in a direction parallel to the axis of the alpha helix. Likewise, two or more negatively charged amino acid residues can form a negatively charged domain.

Antimicrobial, as used herein to describe the polypeptides of the present invention, means that the polypeptide has the capacity to kill, disrupt or inhibit reproduction, or otherwise disable microbial growth so that the polypeptide has a minimal inhibitory concentration (“MIC”) of less than 250 μM, preferably less than 50 μM, more preferably less than 20 μM. The procedures for determining MIC of an antimicrobial polypeptide are known to those skilled in the art and are described in the Materials and Methods section below and in Powell et al. 1995. It is contemplated that, for purposes of the present invention, a polypeptide is an antimicrobial if it has the aforementioned MIC with respect to any microbe. Microbes, as used herein, include fungi, such as Cryphonectria parasitica, Fusarium oxysporum, Septoria musiva, bacteria, such as Agrobacterium tumefaciens, Erwinia amylovoria, and Pseudomonas syringae, particularly, bacteria related to bacterial infections and contamination problems in fresh producing, fruits and vegetables, such as Escherichia coli (E. coli) and Salmonella, as well as mycoplasmae, viruses, viroids, nematodes, protozoa, and the like.

The term “corresponding to” or “having” or “as shown in” or “consisting of” when used in conjunction with a SEQ ID NO for an amino acid sequence refers to an amino acid sequence which is substantially the same amino acid sequence or derivatives thereof. Amino acid additions, deletions, and/or substitutions which do not negate the ability of the peptide to be antimicrobial are within the scope of an amino acid sequence corresponding to or having or as shown in or consisting of a particular amino acid sequence. Such additions, deletions, and/or substitutions can be, for example, the result of point mutations in the DNA encoding the amino acid sequence, such point mutations made according to methods known to those skilled in the art. Substitutions may be conservative substitutions of amino acids. Two amino acid residues are conservative substitutions of one another, for example, where the two residues are of the same type. In this regard, lysine, arginine, and histidine, all of which are positively charged residues, are of the same type. The weakly hydrophobic amino acids alanine, valine, isoleucine, glycine, cysteine, phenylalanine, tryptophan, and proline, and the strongly hydrophobic amino acids leucine and methionine, all of which are nonpolar amino acid residues, are of the same type. Another type of residue is the uncharged polar amino acid residue, which includes serine, threonine, tyrosine, asparagine, and glutamine. Yet another type of residue is the negatively charged amino acid residue, which includes aspartic acid and glutamic acid. Further descriptions of the concept of conservative substitutions are given by French and Robson 1983, Taylor 1986, and Bordo and Argos 1991.

As indicated above, the polypeptide used for the present invention can consist of between 10 and 20 amino acid residues. Where the number of amino acid residues in the polypeptide is q, suitable polypeptides include those where amino acid residues number n, n+7, and n+14 are each a positively charged amino acid. n can be any integer between 1 and q−14, inclusive. For example, nucleic acid molecules encoding polypeptides having positively charged amino acids at residues number 1, 8, and 15; 2, 9, and 16; 3, 10, and 17; 4, 11, and 18; or 5, 12, and 19, are within the scope of the present invention.

Alternatively, the present invention relates to polypeptides whose amino acid residues number n, n+7, and n+10 are each positively charged where n can be any integer between 1 and q−10, inclusive, and q is the number of residues in the polypeptide. Thus, for example, nucleic acid molecules encoding polypeptides having positively charged amino acids at residues number 1 , 8, and 11; or 2, 9, and 12; or 3, 10, and 13; or 4, 11, and 14; or 5, 12, and 15; or 6, 13, and 16; or 7, 14, and 17; or 8, 15, and 18; or 9, 16, and 19; or 10, 17, and 20 are also within the scope of the present invention.

The positively charged residues referred to above can be the same or different and are selected from the group consisting of lysine, arginine, and histidine. The remaining amino acid residues of the antimicrobial polypeptide are not critical to the practice of the present invention so long, of course, that their selection does not preclude the formation of an amphipathic alpha helix secondary structure. It is preferred, however, that at least nine amino acids of the polypeptide are nonpolar amino acids. The at least nine nonpolar amino acids of the polypeptide can be the same or different and are selected from the group consisting of alanine, valine, leucine, isoleucine, glycine, cysteine, phenylalanine, tryptophan, proline, and methionine. In a preferred embodiment, the antimicrobial polypeptide has a methionine as an N-terminal amino acid and at least seven of the nonpolar amino acids are alanine.

In one embodiment, the present invention is direct to a method for disinfecting or sterilizing an object comprising applying to the object at least one antimicrobial polypeptide (AMP), preferably, the antimicrobial polypeptide has an amino acid sequence as shown in SEQ ID NO:1. In other alternative embodiments of the present invention, suitable AMP include, but are not limited to, an amino acid sequence as shown in SEQ ID NO:2, SEQ ID NO:3, SEQ ID NO:4, SEQ ID NO:5, SEQ ID NO:6, SEQ ID NO. 7, SEQ ID NO:8, SEQ ID NO:9, SEQ ID NO:10, SEQ ID NO:11, SEQ ID NO:12, SEQ ID NO:13, SEQ ID NO. 14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28; and SEQ ID NO:29. In other embodiments, the invention contemplates compositions of two or more AMPs.

In another embodiment, the present invention is directed to a method for disinfecting or sterilizing an object comprising applying to the object at least one AMP, wherein the amino acid sequence of the AMP is a polypeptide which comprises an amino acid sequence of between 10 and 20 amino acid residues, four of which, namely n, n+7, n+10, and n+14, are positively charged amino acids, at least one of these positively charged amino acids being arginine. n, as used to describe the particular amino acid residue in the polypeptide, can be any integer from 1 to 1+q, where q is defined by the length of the amino acid sequence and can be 0, 1, 2, 3, 4, 5, 6, or 7. For example, when the amino acid sequence contains 20 amino acid residues, q is 5, and n can be 1, 2, 3, 4, 5, or 6. Thus, when q is 5, polypeptides of the present invention include those having positively charged amino acids at residues number 1, 8, 11, and 15 (n=1); 2, 9, 12, and 16 (n=2); 3, 10, 13, and 17 (n=3); 4, 11, 14, and 18 (n=4); 5, 12, 15, and 19 (n=5); or 6, 13, 16, and 20 (n=6). The remaining amino acid residues of the amino acid sequence are nonpolar amino acids or uncharged polar amino acids.

In this embodiment, that is, where n, n+7, n+10, and n+14 are positively charged amino acids, each positively charged amino acid residue is preferably arginine. In addition, when n is 2, residue number 1 is preferably an uncharged polar amino acid, and, when n is 3 or greater, residue number 1 is preferably a strongly hydrophobic nonpolar amino acid and residue number n−1 is an uncharged polar amino acid. In either case, residues number n+4, n+8, n+11, n+13, and, if q−n is greater than or equal to 1, residue number n+16, are strongly hydrophobic nonpolar amino acids. The remaining amino acid residues are preferably weakly hydrophobic nonpolar amino acids. For example, when q is 5 and n is 4, each of the positively charged amino acids can be an arginine, residue number 1 can be a methionine, residue number 3 can be a serine, residue number 7 can be a glycine, each of residues number 8, 12, 15, 17, and 20 can be a leucine, and each of the remaining amino acids can be an alanine (see SEQ ID NO:3).

In still another embodiment, the present invention is directed to a method for disinfecting or sterilizing an object comprising applying to the object at least one AMP, wherein the amino acid sequence of the AMP is an antimicrobial polypeptide, which polypeptide comprises an amino acid sequence having between 10 and 20, inclusive, amino acid residues. Of these amino acid residues, 6 are positively charged amino acids. More particularly, the positively charged amino acids are residues number n, n+6, n+7, n+10, n+13, and n+14. n can be any integer from 1 to 1+q, where q is defined by the length of the amino acid sequence and can be 0, 1, 2, 3, 4, 5, 6, or 7. Thus, n can be any integer in the range from 1 to 6, inclusive. In one example, the amino acid sequence contains 20 amino acid residues so q is 5 and n can be 1, 2, 3, 4, 5, or 6. When q is 5, nucleic acid molecules encoding polypeptides having amino acid sequences containing positively charged amino acid residues at 1, 7, 8, 11, 14, and 15 (n=1); or 2, 8, 9, 12, 15, and 16 (n=2); or 3, 9, 10, 13, 16, and 17 (n=3); or 4, 10, 11, 14, 17, and 18 (n=4); or 5, 11, 12, 15, 18, and 19 (n=5); or 6, 12, 13, 16, 19, and 20 (n=6) are nucleic acid molecules illustrative of this embodiment.

Where the positively charged amino acid residues are n, n+6, n+7, n+10, n+13, and n+14, and when n is 2, residue number 1 is preferably an uncharged polar amino acid. On the other hand, when n is 3 or 4, it is preferred that residue number 1 be a strongly hydrophobic nonpolar amino acid and that residue number n−1 be an uncharged polar amino acid. In either case, residues number n+4, n+8, and n+11 are preferably strongly hydrophobic nonpolar amino acids, and the remaining amino acids are preferably weakly hydrophobic nonpolar amino acids. For example, in a preferred embodiment where q is 5 and n is 4, the positively charged amino acids are arginine, residue number 1 is methionine, residue number 3 is serine, residues number 8, 12, 15, and 20 are leucine, residue number 7 is glycine, and the remaining amino acid residues are each alanine (see SEQ ID NO:10).

In another yet another embodiment, the present invention is directed to a method for disinfecting or sterilizing an object comprising applying to the object at least one AMP, wherein the amino acid sequence of the AMP is a polypeptide which comprises an amino acid sequence of between 10 and 20 amino acid residues, three of which, namely n, n+7, and n+14, are positively charged amino acids, at least one of these positively charged amino acids being arginine. n, as used to describe the particular amino acid residue in the polypeptide, can be any integer from 1 to 1+q, where q is defined by the length of the amino acid sequence, that is, q can be 0, 1, 2, 3, 4, 5, 6, or 7. For example, when the amino acid sequence contains 20 amino acid residues, q is 5, and n can be 1, 2, 3, 4, 5, or 6. Thus, for example, when q is 5, polypeptides of the present invention include those having positively charged amino acids at residues number 1, 8, and 15 (n=1); 2, 9, and 16 (n=2); 3, 10, and 17 (n=3); 4, 11, and 18 (n=4); 5, 12, and 19 (n=5); or 6, 13, and 20 (n=6). The remaining amino acid residues of the amino acid sequence are nonpolar amino acids or uncharged polar amino acids.

In this embodiment, that is, where residues number n, n+7, and n+14 are positively charged amino acids, it is preferred that residues number n+4, n+8, n+11, and n+13 be strongly hydrophobic nonpolar amino acids. When n is an integer from 2 to 4, residue number 1 is preferably a strongly hydrophobic nonpolar amino acid, and the remaining amino acids are preferably weakly hydrophobic nonpolar amino acids. For example, when q is 5 and n is 4, each of the positively charged amino acids can be arginine, residue number 1 can be methionine, each of residues number 8, 12, 15, 17, and 20 can be leucine, residue number 7 can be glycine, and each of the remaining amino acids can be alanine (see SEQ NO:13).

In still yet another embodiment, the present invention is directed to a method for disinfecting or sterilizing an object comprising applying to the object at least one AMP, wherein the amino acid sequence of the AMP is a polypeptide comprise an amino acid sequence of between 10 and 20 amino acid residues, three of which, namely n, n+7, and n+10, are positively charged amino acids. Preferably, at least one of the three positively charged amino acids is arginine. n, as used to describe the particular amino acid residue in the polypeptide, can be any integer from 1 to 5+q, where q is defined by the length of the amino acid sequence, that is, q can be 0, 1, 2, 3, 4, 5, 6, or 7. For example, when the amino acid sequence contains 20 amino acid residues, i.e., when q is 5, n can be any integer from 1 to 10, inclusive. Thus, for example, when q is 5, polypeptides of the present invention include those containing amino acid sequences having positively charged amino acids at residues number 1, 8, and 11 (n=1); 2, 9, and 12 (n=2); 3, 10, and 13 (n=3); 4, 11, and 14 (n=4); 5, 12, and 15 (n=5); 6, 13, and 16 (n=6); 7, 14, and 17 (n=7); 8, 15, and 18 (n=8); 9, 16, and 19 (n=9); or 10, 17, and 20 (n=10). The remaining amino acid residues of the amino acid sequence are preferably either nonpolar amino acids or uncharged polar amino acids. More particularly, when q is 0 and n is 2, residue number 1 is preferably an uncharged polar amino acid; each of residues number 6, 13, and 15 is a strongly hydrophobic nonpolar amino acid; and each of the remaining residues are weakly hydrophobic amino acids. Especially preferred are polypeptides containing amino acid sequences where q is 0 and n is 2 each of the positively charged amino acids is arginine, residue number 1 is serine; each of residues number 6, 10, 13, and 15 is leucine, residue number 5 is glycine, and each of the remaining amino acids is alanine (see SEQ ID NO:14).

In most cases, the antimicrobial polypeptides used in the present invention are not known to occur in nature. Consequently, DNA molecules encoding the antimicrobial polypeptides can be designed and constructed. Optimal expression of the antimicrobial polypeptides requires taking into account several DNA molecule design considerations. These considerations are discussed below in the Materials and Methods section in the context of designing nucleic acid molecules encoding ESF12 (SEQ ID NO:1) as well as in publications, e.g., U.S. Pat. Nos. 5,856,127 and 5,643,876, which are hereby incorporated by reference in their entirety.

The antimicrobial polypeptides of the present invention can be advantageously produced by either chemical synthesis or by one or more methods of inserting specific nucleic acid molecules encoding one or more of the polypeptides into a host cell and allowing that cell to express the desired polypeptide. With regard to traditional chemical synthesis, antimicrobial polypeptides in accordance with the present invention can be synthesized using any of the known peptide synthesis protocols such as those described in Gross and Meienhofer (1980) and Udenfriend and Meienhofer (1987). Further details relating to the chemical synthesis of the antimicrobial peptides of the present invention can be found, for example, in U.S. Pat. No. 5,519,115 to Mapelli et al. Alternatively, as indicated above, the antimicrobial polypeptides can be produced and isolated by common technology using host cells into which the nucleic acid molecules encoding the antimicrobial polypeptide have been introduced. The host cells are then cultured under conditions effective to functionally express the antimicrobial polypeptide, and subsequently, the antimicrobial polypeptide is isolated from the culture. Alternatively, the antimicrobial peptide can be produced in vivo in the host of interest, where its expression provides the desired antimicrobial protection of the host.

According to the present invention, the antimicrobial polypeptides can have wide use in inhibiting the growth or survival of, or killing microbes, in particular, as disinfectants and sterilization agent for food, particularly, fresh produce, fruits and vegetable, as well as surface areas for food. The antimicrobial polypeptides of the present invention can be useful in all of these contexts either by themselves or in combination with other chemical or pharmaceutical compounds which are effective against microbial pathogens of humans, animals, or plants. For example, the peptides of the invention can be employed in combination with e.g. chlorine bleach for detergent use to effectively disinfect and/or sterilize clothing that may contain unwanted bacteria or fungi or the like. The AMP of the present invention can also be combined with conventional cleaning/disinfecting agents to enhance the efficacy of such agents.

The AMP used in the present invention can be effectively applied to food, particularly, fresh produce, fruits and vegetable, as well as surface areas for food, afflicted with microbes, such as bacteria, fungi, and the like, by any convenient means, including spray, dust, rinse, wash, wipe, dip, or other formulations common to the antimicrobial arts.

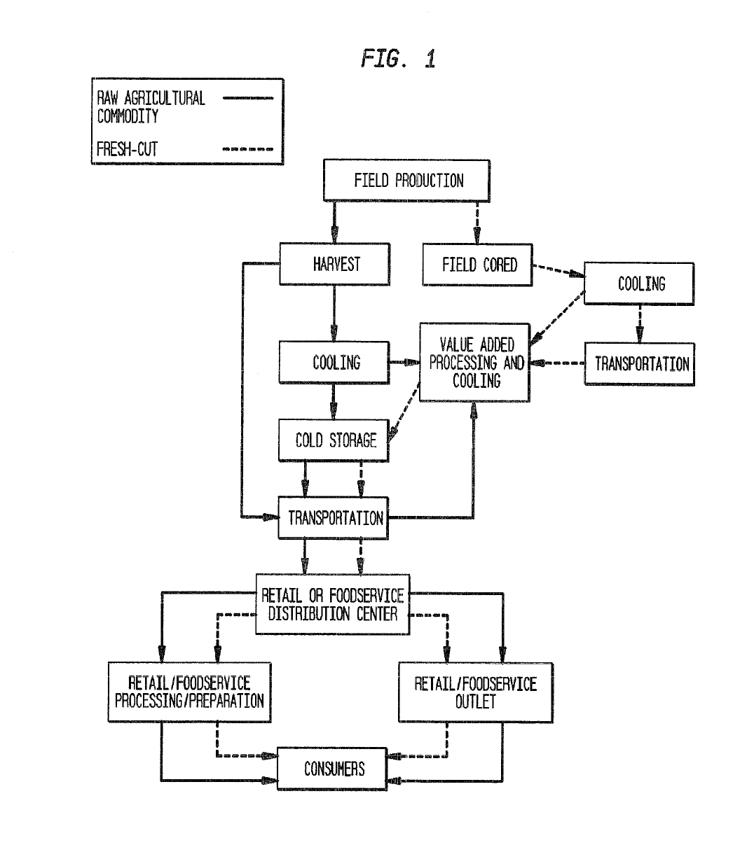

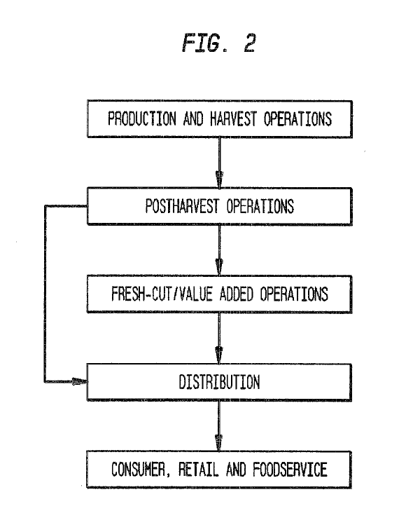

In accordance with the present invention, the AMP can be applied in each step or stage of food processing as shown in FIGS. 1 and 2.

The AMP of the present invention can also be conventionally formulated as a quick-dissolve tablet, which when added to potable water, can thereby render the water safer for human consumption. The tablets of the present invention are prepared by blending the AMP with e.g a hydrophobic material and lubricant, if present, a filler, if present, and any adjuvants, if present. The ingredients are mixed in a typical blender that is normally utilized in the pharmaceutical arts, such as a Hobart mixer, V-blender, a planetary mixer, Twin shell blender and the like. The ingredients are blended together typically at about ambient temperature; no additional heating is necessary, although slight modifications of temperature therefrom could be utilized. It is preferred that the blending be conducted at temperatures ranging from about 10° C. to about 35° C. The hydrophobic material is not melted. Moreover, if the components are heated, the elevated temperature used in the present process is not even substantially close to the melting point of the hydrophobic material. The composition of the present invention is not prepared by thermal infusion.

The ingredients in the formulation are preferably mixed together in a large batch using techniques well known in the pharmaceutical arts and are intimately intermixed until the mixture is homogenous with respect to the drug. The term “homogenous” with respect to the AMP is used to denote that the various components are substantially uniform throughout the invention, i.e., a substantially homogeneous blend is formed.

When the mixture is homogeneous the unit dosage form is prepared by techniques known in the art. Thus, the mixture may be made into a dissolvable pellet, capsule, granule, pill, tablet or other unit dosage form using conventional techniques known in the art.

The preferred unit dosage form is a tablet. The tablet can be prepared by the following procedure.

A unit dosage amount of the homogenous mixture is compressed into a tablet form using a tablet machine typically utilized in the pharmaceutical arts. More specifically, the mixture is fed to the die of a tablet press and sufficient pressure is applied to form a solid tablet. Such pressure can vary, and typically ranges from about 1,000 psi to about 6,000 psi and preferably about 2,000 psi force. The solid formulation according to the present invention is compressed to a sufficient hardness to prevent the premature ingress of the aqueous medium into the tablet. Preferably, the formulation is compressed into a tablet form which is of the order of 5-20 Kp and more preferably 8-20 Kp as determined by a Schleuniger hardness tested.

In a variation, all of the above steps are repeated, except that the mixing is initially performed in the absence of a lubricant, if a lubricant is to be added to the formulation. When the mixture is homogeneous with respect to the AMP, then the lubricant is added and the mixing is continued until the lubricant, if present, may be substantially evenly dispersed in the mixture. Then the mixing is terminated, and the mixture is immediately thereafter made into a unit dosage form. For example, it may be compressed into a tablet, as described hereinabove.

Prior to the mixing step, the individual components may optionally be milled, e.g., passed through a screen, sieve, etc. to reduce the size of the particles thereof. Alternatively, the substantially uniformly blended mixture prior to the formation of the unit dosage form. For example, if it is a tablet, the mixture may be milled before the compression step.

The AMP-containing tablets would comprise about 3 mg to about 30 mg of AMP for use in an 8 oz glass of water, for example.

Alternatively, the AMPs can be readily formulated into powders and sachets, individually packaged or prepared in bulk-containers for use in delivering AMPs to individual glasses of drinking water or municipal water supplies to eradicate unwanted microbes safely and rapidly.

The AMPs of the present invention can be formulated in liquids and sprays as micelles or other conventional delivery forms permitting the AMP to be suspended in micro or nanoparticulate fashion in e.g. water or other conventional excipient prior to delivery to an object's surface, such as a fruit or vegetable.

In addition, the invention contemplates AMPs which can be suitably formulated as ointments, creams or coatings for use alone and/or on bandages for wound healing purposes. Alternatively, the AMPs of the present invention can be applied directly to wounds. In one embodiment, the amount of AMP that would be applied to a wound or bandage would be from about 0.01 μg/kg to about 5 g/kg.

A kit for disinfecting or sterilizing fresh produce, fruits or vegetables, water as well as surface areas for food, comprising at least one AMP and a tool for application is also contemplated by the present invention. The tool can be any suitable means for application of AMP, such as a sprayer, duster, or wiping tissue.

The following examples are offered by way of illustration, not limitation, of the present invention.

### Materials and Methods

### Peptide Design

All peptides were designed so that they had an .alpha.-helical conformation as predicted by the Chou-Fasman (Chou and Fasman 1978 and Chou 1990) or Garnier-Robson (Garnier et al. 1978) algorithms. The presence of an amphipathic region was predicted by the Eisenberg Moment (Eisenberg et al. 1984). The PROTEAN program from DNASTAR, Inc. (Madison, Wis.) was employed for these analyses.

### Peptide Synthesis

Six antimicrobial polypeptides, designated ESF1 (SEQ ID NO:3), ESF1B (SEQ ID NO:7), ESF5 (SEQ ID NO:10), ESF6 (SEQ ID NO:13), ESF12 (SEQ ID NO:1), ESF17 (SEQ ID NO:4), and ESF15 (SEQ ID NO:14) were designed in accordance with the above considerations. Two other ESF peptides, ESF4 and ESF13, contained uncharged polar and negative amino acid substitutions for the positive amino acids in the ESF1 sequence, respectively (see Powell et al. 1995). These were used to test the necessity of the positive charges. All sequences were synthesized and purified to greater than 80% purity by Genosys Biotechnologies (The Woodlands, Tex.). Magainin 11, (Ala.sup.8,13,18)Magainin 11 amide, and cecropin B (HPLC purified to 97%) were purchased from Sigma (St. Louis, Mo.).

### Determination of the Minimal Inhibitory Concentration

Dilutions of peptides, prepared in sterile deionized water, were placed (20 μl aliquots) in Corning disposable sterile polystyrene ELISA plates (96 well, high binding) which had been pretreated with bovine serum albumin by rinsing each well with 200 μl of a 1.0 mg/ml solution.

Media (PDAmb, pH 5.2) was prepared with deionized water, Difco potato dextrose broth (12 g/L), Sigma D,L-methionine (0.05 g/L), Sigma biotin (2 mg/L), and FisherBiotech low melting point agarose (20 g/L). Sterilized media was added to the wells to make a final volume of 100 μl and allowed to harden for 3 hr prior to inoculation with 10 μl of conidial suspension. Peptide concentrations tested were 0, 1.25, 2.5, 5, 10, 15, 20, 25, 50, 100, 150, 200, and 250 μm.

Fungal conidial suspensions were aseptically prepared from agar plate cultures of C. parasitica and S. musiva or liquid cultures for F. oxysporum f.sp. lycopersici. Conidia were suspended from agar plates with 10 ml sterile 1% Tween 20. All culture suspensions were filtered through four layers of sterile cheesecloth, collected in sterile 15 ml Falcon tubes, and centrifuged for 3 min at 1900×g. The pellets were then suspended in 10 ml sterile deionized water and centrifuged for 3 min at 1900×g. The “washed” pellets were suspended in 5 ml sterile deionized water. The conidial concentration was determined using a hemocytometer (Fisher ultra plane, Neubauer ruling) and then diluted to 1.0×10⁴ conidia/ml.

Approximately 100 conidia (10 μl) were transferred to each microtitre plate well. Microtitre plates were incubated in ambient light at room temperature in a moist chamber (plastic box containing wet paper towels and covered with clear plastic wrap). Tests with S. musiva used continuous light. Plates were scored for growth at 6 days and photographed. All tests were repeated 4 or more times. Controls without peptides were included with all tests. The Minimum Inhibitory Concentration (“MIC”) of the fungal conidia was the lowest peptide concentration which totally prevented germination on the medium in all the repeated tests.

Bacterial suspensions of A. tumefaciens, E. amylovora, and P. syringae were obtained from liquid cultures grown in Luria Broth (Atlas and Parks 1993). Cultures were grown at 25° C. on a rotary shaker (110 rpm) for 14 hr. Bacterial cell suspensions with an optical density between 0.7 and 0.85 at 600 nanometers (OD₆₀₀) were used for assay inoculations. Dilutions of peptides were placed in the 96-well ELISA plates as described above. The final peptide concentrations tested were 0, 0.625, 1.25, 2.5, 5, 10, 15, 20, 25, 50, 100, and 250 μM. All tests were repeated 2 or more times. Controls without peptides were included with all tests. The MIC for bacterial cells was the lowest peptide concentration associated with no visible bacterial lawn or isolated colonies on the medium after 3 days in all the repeated tests.

Rehydrated plant pollen was diluted with sucrose-boric acid medium (“SBM”) to a concentration of 80 grains/μl (Neubauer hemocytometer). Media for pollen germination (SBM, pH 6.2) was prepared with deionized distilled water, sucrose (150 g/L), and boric acid (150 mg/L). Dilutions of all peptides tested against rehydrated pollen grains were aseptically prepared with sterile SBM. Aliquots (50 μl) of peptide solutions were aseptically placed in ELISA microtitre plates as before. Approximately 400 pollen grains (5 μl) were added to the 50 μl volume of peptide solution in the well to give the final peptide concentrations of 0, 1.25, 2.5, 5, 10, 15, 20, 25, 50, 100, 150, 200, and 250 μM. Microtitre plates were incubated in the dark at room temperature for 24 hr. Germination of pollen grains was observed with Zeiss stereo dissecting scope (Schott cold light source) and photographed. The MIC of the plant pollen was the lowest concentration that totally inhibited germination in the medium. All tests were repeated 4 or more times. Controls without peptides were included with all tests. The highest concentration tested was 250 μM. If germination occurred at this concentration, the MIC is yet undetermined, but it is known to be greater than 250 μM.

It should be noted that in view of the above-described method, the MICs may need to be determined under conditions of use for the purpose of the present application. For example, if AMP are used in combination with a wetting agent, the MIC may be different. According to the present invention, the MICs will have to be tested for each formulation.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

### LIST OF REFERENCES CITED

Agerberth et al., Eur. J. Biochem., 202:849-854 (1991).
Anagnostakis, S. L., Mycologia, 74:826-830 (1982).
Angenon, G. et al., FEBS Letters, 271:144-146 (1990).
Atlas, R. M. and Parks, L. C., Microbiological Media., Boca Raton: CRC Press (1993).
Bevins, C. L. and Zasloff, M., Annu. Rev. Biochem. 59:395-414 (1990).
Blondelle, S. E. and Houghten, R. A., Biochemistry, 30:4671-4678 (1991).
Boman, H. G. et al., FEBS Letters, 259:103-106 (1989).
Bordo, D. and Argos, P., J Mol Biol 217:721-729 (1991).
Broekaert, W. F., et al., Biochemistry, 31:4308-4314 (1992).
Campbell, W. H. and Gowri, G., Plant Physiol., 92:1-11 (1990).
Cao, J. et al., Plant Cell Rep, 11:586-591 (1992).
Chen, H.-C. et al., FEBS Letters, 236:462-466 (1988).
Chou, P. Y. and Fasman, G. D., Adv. Enzymol., 47:45-148 (1978).
Chou, P. Y., Prediction Of Protein Structure And The Principles Of Protein Conformation, New York: Plenum Press, 549-586 (1990).
Christensen, B. et al., Proc. Natl. Acad. Sci. USA, 85:5072-5076 (1988).
Coico, R., ed, Current Protocols in Immunology, Vol. 1, New York: John Wiley & Sons, Inc., pp. 2.4.1-2.4.4 (1995).
Cornelissen, B. J. C. and Melchers, L. S., Plant Physiol., 101:709-712 (1993).
Crowther, J. R., Methods in Molecular Biology, New Jersey: Humana Press, 42:223 (1995).
Davis, J. M. et al., Plant Mol. Biol., 17:631-639 (1991). de Niella, P. R. and Maynard, C. A., “Storage of Chestnut and Willow Pollen,” In: Mohn, C. A. (Complier) Proceedings of the Second Northern Forest Genetics Association Conference, Jul. 29-30, 1993, Roseville, Minn., St. Paul, Minn.: Northern Forest Genetics Association, Department of Forest Resources, University of Minnesota, pp. 171-180 (1993).
Denecke, J. et al., Plant Cell, 2:51-59 (1990).
Ditta, G. et al., Proc. Natl. Acad. Sci. USA, 77:7347-7351 (1981).
Eisenberg, D. et al., Proc. Natl. Acad. Sci. USA, 81(1):140-144 (1984).
French, S, and Robson, B., J. Molecular Evolution 19:171-175 (1983).
Galvin, S. B. and Schilperoort, R. A., eds., Plant Molecular Biology Manual, 2nd ed., Dordrecht, Netherlands: Kluwer Academic Press (1994).
Garnier, J. et al., J. Mol. Biol., 120:97-120 (1978).
Gross, E. and Meinhofer, J., eds., The Peptides: Analysis, Synthesis, Biology. Special Methods in Peptide Synthesis, Part A, New York: Academic Press (1980).
Guerineau, F. et al., Plant Mol. Biol., 18:815-818 (1992).
Hiei, Y. et al. The Plant Journal, 6:271-282 (1994).
Hollick, J. B. and Gordon, M. P., Plant Mol. Biol., 22:561-572 (1993).
Holsters, M. et al., Mol. Gen. Genet., 163:181-187 (1978).
Horsch, R. B. et al., Science, 227:1229-1231 (1985).
Joshi, C. P., Nucleic Acids Res., 16:6643-6653 (1987).
Kozak, M., Proc. Natl. Acad. Sci. USA, 83:2850-2854 (1986a).
Kozak, M., Cell, 44:283-292 (1986b).
Mackey, C. J. et al., Transgenic Plants, 2:21-33 (1993).
Melchers, L. S. et al., Plant Molecular Biology, 21:583-593 (1993).
Pfitzner, U. M. and Goodman, H. M., Nucleic Acids Res., 15:4449-4465 (1987).
Powell, W. A. et al., Molecular Plant-Microbe Interactions, 8:792-794 (1995).
Putterill, J. J. and Gardner, R. C., Plant Science, 62:199-205 (1989).
Sambrook et al., Molecular Cloning: A Laboratory Manual, 2d Edition, Cold Spring Harbor, N.Y.: Cold Spring Harbor Laboratory Press (1989).
Sciaky, D. et al., Plasmid, 1:238-253 (1978).
Shen, W. and Forde, B. G., Nucleic Acids Res., 17:8385 (1989).
Simon, R. et al., Biotechnology, 1:784-791 (1982).
Soravia, E. et al., FEBS Letters, 228:337-340 (1988).
Studier et al., Gene Expression Technology, 185 (1990).
Taylor, W. R., J. Theor. Biol. 119:205-218 (1986).
Udenfriend, S, and Meinhofer, J., eds., The Peptides: Analysis, Synthesis, Biology. Vol. 9: Special Methods in Peptide Synthesis, Part C, San Diego: Academic Press (1987).
Wade, D. et al., Biochemistry, 87:4761-4765 (1990).
Zasloff, M., Proc. Natl. Acad. Sci. USA, 84:5449-5453 (1987).

What is claimed:

1. A method for disinfecting or sterilizing an object comprising applying to the object at least one antimicrobial polypeptide (AMP), wherein the amino acid sequence of the AMP is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28; and SEQ ID NO:29.

2. A method for disinfecting or sterilizing an object comprising applying to the object at least one AMP, wherein the amino acid sequence of the AMP is selected from the group consisting of:
a polypeptide comprising an amino acid sequence of 13+q amino acid residues, wherein residues number n, n+7, n+10, and n+14 are positively charged amino acids, wherein at least one of residues number n, n+7, n+10, and n+14 is arginine, wherein remaining amino acid residues are nonpolar amino acids or uncharged polar amino acids, wherein n is an integer from 1 to 1+q, and wherein q is 0, 1, 2, 3, 4, 5, 6, or 7;
a polypeptide comprising an amino acid sequence of 13+q amino acid residues, wherein residues number n, n+6, n+7, n+10, n+13, and n+14 are positively charged amino acids, wherein remaining amino acid residues are nonpolar amino acids or uncharged polar amino acids, and wherein n is an integer from 1 to 1+q, and wherein q is 0, 1, 2, 3, 4, 5, 6, or 7;
a polypeptide comprising an amino acid sequence of 13+q amino acid residues, wherein residues number n, n+7, and n+14 are positively charged amino acids, wherein remaining amino acid residues are nonpolar amino acids, wherein n is an integer from 1 to 1+q, and wherein q is 0, 1, 2, 3, 4, 5, 6 or 7;
a polypeptide comprising an amino acid sequence of 13+q amino acid residues, wherein residues number n, n+7, and n+10 are positively charged amino acids, wherein remaining amino acid residues are nonpolar amino acids, wherein n is an integer from 1 to 5+q, and wherein q is 0, 1, 2, 3, 4, 5, 6, or 7;
a polypeptide comprising an amino acid sequence of 13+q amino acid residues, wherein residues number n, n+3, n+7, n+10, and n+14 are positively charged amino acids, wherein at least one of residues number n, n+3, n+7, n+10, and n+14 is arginine, wherein remaining amino acid residues are nonpolar amino acids or uncharged polar amino acids, wherein n is an integer from 1 to 1+q, and wherein q is 0, 1, 2, 3, 4, 5, 6, or 7;
a polypeptide comprising an amino acid sequence of 13+q amino acid residues, wherein residues number n, n+3, n+6, n+7, n+10, n+13, and n+14 are positively charged amino acids, wherein remaining amino acid residues are nonpolar amino acids or uncharged polar amino acids, and wherein n is an integer from 1 to 1+q, and wherein q is 0, 1, 2, 3, 4, 5, 6, or 7;
a polypeptide comprising an amino acid sequence of 13+q amino acid residues, wherein residues number n, n+3, n+7, and n+14 are positively charged amino acids, wherein remaining amino acid residues are nonpolar amino acids, wherein n is an integer from 1 to 1+q, and wherein q is 0, 1, 2, 3, 4, 5, 6, or 7; and
a polypeptide comprising an amino acid sequence of 13+q amino acid residues, wherein residues number n, n+3, n+7, and n+10 are positively charged amino acids, wherein remaining amino acid residues are nonpolar amino acids, wherein n is an integer from 1 to 5+q, and wherein q is 0, 1, 2, 3, 4, 5, 6, or 7.

3. The method of claim 2, wherein the polypeptide has a methionine residue as an N-terminal amino acid.

4. The method of claim 2, wherein the positively charged amino acids are the same or different and are selected from the group consisting of lysine, arginine, and histidine.

5. The method of claim 2, wherein the nonpolar amino acids are the same or different and are selected from the group consisting of alanine, valine, leucine, isoleucine, glycine, cysteine, phenylalanine, tryptophan, and methionine.

6. The method of any one of claims 1-5, wherein the object is fresh produce, fruits or vegetables.

7. The method of claim 6, wherein the vegetable is green leafy vegetable.

8. The method of any one of claims 1-5, wherein the microbe is bacterium.

9. The method of claim 8, wherein the bacterium is E. coli.

10. The method of claim 1, wherein the object is a surface area fresh produce, fruits or vegetables.

11. The method of any one of claims 1-5 and 10, wherein the AMP is applied to the object by means of spraying, washing, rinsing, wiping, dusting, or dipping.

12. A kit for disinfecting or sterilizing fresh produce, fruits or vegetables comprising at least one AMP and a tool for application.

13. An isolated AMP selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28; and SEQ ID NO:29.
